SEQUENCE LISTING (13 sequences)

SEQ ID NO: 1 - DNA, 2100 bp, Homo sapiens
atgtcgccatttcttcggattggcttgtccaactttgactgcgggtcctgccagtcttgtcagggcgaggctgttaacccttactgtgctgtgctcgtcaaagagtatgtcgaatcagagaacgggcagatgtatatccagaaaaagcctaccatgtacccaccctgggacagcacttttgatgcccatatcaacaagggaagagtcatgcagatcattgtgaaaggcaaaaacgtggacctcatctctgaaaccaccgtggagctctactcgctggctgagaggtgcaggaagaacaacgggaagacagaaatatggttagagctgaaacctcaaggccgaatgctaatgaatgcaagatactttctggaaatgagtgacacaaaggacatgaatgaatttgagacggaaggcttctttgctttgcatcagcgccggggtgccatcaagcaggcaaaggtccaccacgtcaagtgccacgagttcactgccaccttcttcccacagcccacattttgctctgtctgccacgagtttgtctggggcctgaacaaacagggctaccagtgccgacaatgcaatgcagcaattcacaagaagtgtattgataaagttatagcaaagtgcacaggatcagctatcaatagccgagaaaccatgttccacaaggagagattcaaaattgacatgccacacagatttaaagtctacaattacaagagcccgaccttctgtgaacactgtgggaccctgctgtggggactggcacggcaaggactcaagtgtgatgcatgtggcatgaatgtgcatcatagatgccagacaaaggtggccaacctttgtggcataaaccagaagctaatggctgaagcgctggccatgattgagagcactcaacaggctcgctgcttaagagatactgaacagatcttcagagaaggtccggttgaaattggtctcccatgctccatcaaaaatgaagcaaggctgccatgtttaccgacaccgggaaaaagagagcctcagggcatttcctgggagtctccgttggatgaggtggataaaatgtgccatcttccagaacctgaactgaacaaagaaagaccatctctgcagattaaactaaaaattgaggattttatcttgcacaaaatgttggggaaaggaagttttggcaaggtcttcctggcagaattcaagaaaaccaatcaatttttcgcaataaaggccttaaagaaagatgtggtcttgatggacgatgatgttgagtgcacgatggtagagaagagagttctttccttggcctgggagcatccgtttctgacgcacatgttttgtacatttcagaccaaggaaaacctcttttttgtgatggagtacctcaacggaggggacttaatgtaccacatccaaagctgccacaagttcgacctttccagagcgacgttttatgctgctgaaatcattcttggtctgcagttccttcattccaaaggaatagtctacagggacctgaagctagataacatcctgttagacaaagatggacatatcaagatcgcggattttggaatgtgcaaggagaacatgttaggagatgccaagacgaataccttctgtgggacacctgactacatcgccccagagatcttgctgggtcagaaatacaaccactctgtggactggtggtccttcggggttctcctttatgaaatgctgattggtcagtcgcctttccacgggcaggatgaggaggagctcttccactccatccgcatggacaatcccttttacccacggtggctggagaaggaagcaaaggaccttctggtgaagctcttcgtgcgagaacctgagaagaggctgggcgtgaggggagacatccgccagcaccctttgtttcgggagatcaactgggaggaacttgaacggaaggagattgacccaccgttccggccgaaagtgaaatcaccatttgactgcagcaatttcgacaaagaattcttaaacgagaagccccggctgtcatttgccgacagagcactgatcaacagcatggaccagaatatgttcaggaacttttccttcatgaacccccgg

SEQ ID NO: 2 - protein, 729 aa, Homo sapiens
MSPFLRIGLSNFDCGSCQSCQGEAVNPYCAVLVKEYVESENGQMYFTIQKKPTMYPPWDSTFDAHINKGRVMQIIVKGKNVDLISETTVELYSLAERCRKNNGKTEFTIWLELKPQGRMLMNARYFLEMSDTKDMNEFETEGFFALHQRRGAIKQAKVHHVKCHEFTFTATFFPQPTFCSVCHEFVWGLNKQGYQCRQCNAAIHKKCIDKVIAKCTGSAINSRETMFHFTKERFKIDMPHRFKVYNYKSPTFCEHCGTLLWGLARQGLKCDACGMNVHHRCQTKVANLCFTINQKLMAEALAMIESTQQARCLRDTEQIFREGPVEIGLPCSIKNEARLPCLPTPGKREFTPQGISWESPLDEVDKMCHLPEPELNKERPSLQIKLKIEDFILHKMLGKGSFGKVFLAEFFTKKTNQFFAIKALKKDVVLMDDDVECTMVEKRVLSLAWEHPFLTHMFCTFQTKENLFFVMFTEYLNGGDLMYHIQSCHKFDLSRATFYAAEIILGLQFLHSKGIVYRDLKLDNILLDKDGHFTIKIADFGMCKENMLGDAKTNTFCGTPDYIAPEILLGQKYNHSVDWWSFGVLLYEMLIGQFTSPFHGQDEEELFHSIRMDNPFYPRWLEKEAKDLLVKLFVREPEKRLGVRGDIRQHPLFRFTEINWEELERKEIDPPFRPKVKSPFDCSNFDKEFLNEKPRLSFADRALINSMDQNMFRNFFTSFMNPRMERLIS

SEQ ID NO: 3 - DNA, 22 bp, Artificial sequence
  Synthetic oligonucleotide (antisense against V3 region of PKC-theta)
tcttgaactctgccaggaagac

SEQ ID NO: 4 - DNA, 20 bp, Artificial Sequence
  Synthetic oligonucleotide (antisense against region C1 of PKC-theta)
atcggtggtggacgttcatg

SEQ ID NO: 5 - DNA, 20 bp, Artificial Sequence
  Synthetic oligonucleotide (antisense sequense against region C1 of PKC-theta)
gcacttcttgtgaatcgctg

SEQ ID NO: 6 - DNA, 20 bp, Artificial Sequence
  Synthetic oligonucleotide (antisense sequence of PKC-theta)
tggttgcgccctggagcgag

SEQ ID NO: 7 - DNA, 20 bp, Artificial Sequence
  Synthetic oligonucleotide (antisense sequence of PKC-theta)
tggcgacatggttgcgccct

SEQ ID NO: 8 - DNA, 20 bp, Artificial Sequence
  Synthetic oligonucleotide (antisense sequence of PKC-theta)
gtagagctccacggtggttt

SEQ ID NO: 9 - DNA, 20 bp, Artificial Sequence
  Synthetic oligonucleotide (antisense sequence of PKC-theta)
tttctgtcttcccgttgttc

SEQ ID NO: 10 - DNA, 20 bp, Artificial Sequence
  Synthetic oligonucleotide (antisense sequence of PKC-theta)
ccagggtgggtacatggtag

SEQ ID NO: 11 - DNA, 20 bp, Artificial Sequence
  Synthetic oligonucleotide (antisense sequence of PKC-theta)
gcatgactcttcccttgttg

SEQ ID NO: 12 - DNA, 20 bp, Artificial Sequence
  Synthetic oligonucleotide (PKC-theta antisense sequence)
caccccggcgctgatgcaaa

SEQ ID NO: 13 - DNA, 20 bp, Artificial Sequence
  Synthetic oligonucleotide (antisense sequence of PKC-theta)
tatcaatacacttcttgtga

(19) United States
(12) Patent Application Publication
(10) Pub. No.: US 2009/0291073 A1
(43) Pub. Date: Nov. 26, 2009

(54) Compositions Comprising PKC-theta and Methods for Treating or Controlling Ophthalmic Disorders Using Same

(76) Inventors: Keith W. Ward, Ontario, NY (US); Srini Venkastesh, Pittsford, NY (US)

Correspondence Address: Bausch & Lomb Incorporated, One Bausch & Lomb Place, Rochester, NY, 14604-2701, US

(21) Appl. No.: 12/123,683

(22) Filed: May 20, 2008

(51) Int. Cl. A61K 31/352; A61P 27/02; A61K 39/395; A61K 31/7088; A61K 31/713; A61K 31/7105; A61K 31/436; A61K 38/13; A61K 31/52

(52) U.S. Cl. 424/130.1; 514/456; 514/291; 514/11; 514/263.22; 514/263.31; 514/44 A

(57) ABSTRACT

Compositions for treating or controlling: (i) an eye condition, disorder, or disease, or (ii) a degeneration of a component of an optic nerve system in a subject, comprise a PKC-θ inhibitor. The compositions can further include an anti-inflammatory or anti-glaucoma medicament. Such a condition or degeneration has an inflammatory component.

### BACKGROUND OF THE INVENTION

The present invention relates to compositions and methods for treating or controlling ophthalmic disorders having an inflammatory component. In particular, the present invention relates to compositions that comprise protein kinase C-θ (“PKC-θ”) and methods for the treatment or control of ophthalmic disorders having an inflammatory component, using such compositions.

Ophthalmic conditions may be classified as front-of-eye diseases, such as corneal edema, anterior uveitis, pterygium, corneal diseases, or opacifications with an exudative or inflammatory component, conjunctivitis, allergy- and laser-induced exudation, or back-of-eye diseases such as exudative macular degeneration, macular edema, diabetic retinopathy, age-related macular degeneration, or retinopathy of prematurity. Back-of-eye diseases comprise the largest number of causes for vision loss. There has been growing evidence that many back-of-the eye diseases have etiology in inflammation.

Among the back-of-the-eye diseases, diabetic retinopathy is the leading cause of blindness in adults between the ages of 18 to 72 who suffer from diabetes. In the early stage, the vasculature of the retina is increasingly obstructed by the adhesion of cells involved in immunological response, such as leucocytes, on molecules, such as intercellular adhesion molecule-1 (“ICAM-1”) or vascular cell adhesion molecule-1 (“VCAM-1”), which are overexpressed on the endothelial layer of inflammed vasculature. The vasculature obstruction results in ischemia and leads to hypoxia condition in the surrounding tissues, especially the retina. In response to such a condition, new blood vessels begin to proliferate uncontrollably. These new blood vessels are typically leaky, resulting in fluid accumulation under the retina and eventually the vision-threatening condition known as macular edema. (See; e.g., A. P. Adamis, British J. Ophthalmol., Vol. 86, 363 (2002); S. Ishida et al., Invest. Ophthalmol. & Visual Sci., Vol. 44, No. 5, 2155 (2003).) Vascular endothelial growth factor (“VEGF”), a hypoxia-induced proinflammatory angiogenic factor, has been found at elevated levels in the diabetic retina during both the nonproliferative and proliferative stage. VEGF also induces the expression of ICAM-1 and VCAM-1 on endothelial cells. (I. Kim et al., Biol. Chem., Vol. 276, No. 10, 7614 (2001).) In addition, experimental investigations in animals have shown that mRNA expression for the proinflammatory cytokines IL-1 (interleukin-1) and TNF-α (tumor necrosis factor-α) is increased in the retina early in the course of diabetes, and moreover, inhibition of TNF-α has demonstrated beneficial effects in the prevention of diabetic retinopathy. (J. F. Navarro and C. Mora, Nephrol. Dial. Transplant, Vol. 20, 2601 (2005).) Thus, experimental evidence strongly suggests a central and causal role of chronic inflammation in the pathogenesis of diabetic retinopathy and macular edema. (A. M. Joussen et al., FASEB J., Vol. 18, 1450 (2004).)

Macular degeneration, another back-of-the-eye degenerative condition, is the most common cause of central vision loss in those 50 or older, and its prevalence increases with age. Age-related macular degeneration (“AMD”) is the more common form of the condition. The other form, which is sometimes called “juvenile macular degeneration” (“JMD”) is most commonly caused by an inherited condition. It is estimated that 50 million people worldwide suffer from AMD. It has recently been discovered that mutations in two genes encoding proteins in the so-called complement cascade account for most of the risk of developing AMD. This complex molecular pathway is the body's first line of defense against invading bacteria, but if overactive, the pathway can produce tissue-damaging inflammation, which underlies the vision-destroying changes that particularly strike the macula. Proteins associated with immune system activity have been found in or near drusen in eyes with AMD. Over time, the drusen grow as they accumulate inflammatory proteins and other materials, and the inflammation persists, causing additional damage to the retina and eventual vision loss. (See; e.g., Science, Vol. 311, 1704 (2006).)

In addition, uveitis is an inflammation inside the eye, affecting one or more parts of the uvea. Anterior uveitis is localized primarily to the anterior segment of the eye and includes iritis (inflammation in the anterior chamber alone) and iridocyclitis (inflammation in the anterior chamber and anterior vitreous). Intermediate uveitis (peripheral uveitis or chronic cyclitis) occurs in the vitreous. Posterior uveitis is a term given to a collection of inflammatory conditions associated with the posterior segment of the eye. Posterior uveitis includes, but is not limited to, choroiditis (inflammation of the choroid), retinitis (inflammation of the retina), optic neuritis (inflammation of the optic nerve), and vasculitis (inflammation of the blood vessels at the back of the eye). Diffused uveitis (panuveitis) is an inflammatory condition of both the anterior and posterior chambers. Ocular complications of uveitis may produce profound and irreversible loss of vision, when unrecognized or treated improperly. The most frequent complications include glaucoma, retinal detachment, neovascularization of the retina or optic nerve, and cystoid macular edema (the most common cause of decreased vision from uveitis). Uveitis is a frequently encountered eye disease because it can result from a bacterial, viral, fungal, or parasitic infection, a systemic disease (such as Crohn's disease or ulcerative colitis), an autoimmune disease (such as rheumatoid arthritis or ankylosing spondylitis), or a trauma to the eye.

Thus, it has been established that the cause of a great number of serious eye diseases may be traced to inflammation.

Glucocorticoids (also referred to herein as “corticosteroids”) have been under investigation for use as a local therapeutic treatment for diabetic retinopathy. (See; e.g., M. A. Speicher et al., Expert Opinion on Emerging Drugs, Vol. 8, No. 1, 239 (2003); E. A. Felinski and D. A. Antonetti, Curr. Eye Research, Vol. 30, No. 11, (49 (2005); and G. M. Corner and T. A. Ciulla, Expert Opinion on Emerging Drugs, Vol. 10, No. 2, 441 (2005).) Intravitreal injection of corticosteroids (especially triamcinolone acetonide) has been investigated as a treatment for diabetic macular edema (see; e.g., V. Vasumathy et al., Ophthalmic Practice, Vol. 54, No. 2, 133 (2006); P. Massin et al., Opthalmology, Vol. 111, No. 2, 218 (2004)). Periocular injection of corticosteroids has been investigated as a treatment for posterior uveitis (see; e.g., Ferrante et al., Clin. Exp. Ophthalmol., Vol. 32, No. 6, 563 (2004). A Phase-III clinical trial of periocular injection of triamcinolone acetonide as adjunct therapy to photodynamic therapy (“PDT”) for neovascular AMD was completed by Johns Hopkins University School of Medicine and Oregon Health Science University in 2006 (see www.clinicaltrials.gov). However, steroidal drugs can have side effects that threaten the overall health of the patient.

It is known that certain glucocorticoids have a greater potential for elevating intraocular pressure (“IOP”) than other compounds in this class. For example, it is known that prednisolone, which is a very potent ocular anti-inflammatory agent, has a greater tendency to elevate IOP than fluorometholone, which has moderate ocular anti-inflammatory activity. It is also known that the risk of IOP elevations associated with the topical ophthalmic use of glucocorticoids increases over time. In other words, the chronic (i.e., long-term) use of these agents increases the risk of significant IOP elevations. Unlike bacterial infections or acute ocular inflammation associated with physical trauma, which requires short-term therapy on the order of a few weeks, many of the conditions discussed above require treatment for extended periods of time, generally several months or more. This chronic use of corticosteroids significantly increases the risk of IOP elevations. In addition, use of corticosteroids is also known to increase the risk of cataract formation in a dose- and duration-dependent manner. Once cataracts develop, they may progress despite discontinuation of corticosteroid therapy.

Chronic administration of glucocorticoids also can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

Therefore, there is a continued need to provide pharmaceutical compounds and compositions to treat, reduce, or ameliorate ocular disorders or conditions, which compounds and compositions cause a lower level of at least an adverse side effect than at least a prior-art glucocorticoid used to treat, reduce, or ameliorate the same condition.

### SUMMARY

In general, the present invention provides pharmaceutical compounds and compositions for treating or controlling in a subject an eye condition or disorder, which has an inflammatory component.

In one aspect, such an inflammatory component comprises a sequela of the eye condition or disorder.

In another aspect, such an inflammatory component comprises an etiology of the eye condition or disorder.

In still another aspect, the compounds and compositions of the present invention cause a lower level of at least an adverse side effect than at least a prior-art glucocorticoid used to treat or control the same condition or disorder.

In yet another aspect, such an eye condition or disorder comprises a back-of-the-eye condition or disorder.

In a further aspect, such an eye condition or disorder comprises a front-of-the-eye condition or disorder.

In yet another aspect, such a condition or disorder is selected from the group consisting of diabetic retinopathy (“DR”), age-related macular degeneration (“AMD,” including dry and wet AMD), diabetic macular edema (“DME”), uveitis (including posterior uveitis, anterior uveitis, intermediate uveitis, panuveitis), corneal inflammatory diseases (such as corneal edema, exudation, or opacification), non-allergic conjunctivitis (such as viral, bacterial, chlamydial, and giant papillary conjunctivitis), keratoconjunctivitis, endophthalmitis, ocular neurodegeneration (including all types of glaucoma), optic neuritis, retinitis pigmentosa, and combinations thereof.

In still another aspect, a composition of the present invention comprises a modulator of PKC-θ, in an effective amount for treating or controlling such an eye condition or disorder.

In yet another aspect, a composition of the present invention comprises a compound that substantially inhibits, reduces, or interferes with, an activity of a cell signaling cascade involving PKC-θ, in an effective amount for treating or controlling such an eye condition or disorder.

In yet another aspect, a composition of the present invention comprises an inhibitor of, or an antagonist to, PKC-θ, or an inhibitor of activation of PKC-θ, in an amount effective for treating or controlling such an eye condition or disorder in a subject. A compound that substantially inhibits, reduces, or interferes with, an activity of a cell signaling cascade involving PKC-θ, antagonizes PKC-θ, or inhibits the activation of PKC-θ hereinafter sometimes referred to as “PKC-θ inhibitor.”

In still another aspect, such PKC-θ is a human PKC-θ.

In still another aspect, such PKC-θ is expressed in a cell or tissue associated with the human ocular system.

In yet another aspect, such PKC-θ is activated in a cell or tissue associated with the human ocular system.

In still another aspect, such a PKC-θ inhibitor is capable of down regulating a cell signaling pathway involving PKC-θ.

In yet another aspect, a composition of the present invention comprises a compound that is capable of inhibiting an activation of a human PKC-θ signaling pathway.

In a further aspect, a composition of the present invention comprises: (a) a PKC-θ inhibitor; and (b) an anti-inflammatory medicament.

In yet another aspect, the present invention provides a method for treating or controlling an eye condition or disorder of a subject. The method comprises administering a composition to an affected eye of the subject, which composition comprises a PKC-θ inhibitor; or a compound that is capable of inhibiting an activation of a human PKC-θ signaling pathway; or a combination thereof; in an effective amount for treating or controlling such eye condition or disorder.

In a further aspect, the present invention provides a method for treating or controlling degeneration of at least a component of an optic nerve system. The method comprises administering a composition to an affected eye, which composition comprises a PKC-θ inhibitor; or a compound that is capable of inhibiting an activation of a human PKC-θ signaling pathway; or a combination thereof; in an effective amount for treating or controlling such degeneration.

Other features and advantages of the present invention will become apparent from the following detailed description and claims.

### DETAILED DESCRIPTION

As used herein, the term “control” also includes reduction, alleviation, amelioration, and prevention.

As used herein, the term “neuroprotection” means the rescue of at least some cells or components of a nervous system that are not directly damaged by the primary cause of a disease or injury, but would otherwise undergo secondary degeneration without therapeutic intervention. In one aspect, neuroprotection can lead to preservation of the physiological function of these cells or components. In one aspect, such a nervous system is the optic nerve system. The cells or components of the optic nerve system include those being involved or assisting in conversion of photon to neurological signal and the transmission thereof from the retina to the brain for processing. Thus, the main cells or components of the optic nerve system include, but are not limited to, pigment epithelial cells, photoreceptor cells (rod and cone cells), bipolar cells, horizontal cells, amacrine cells, interplexiform cells, ganglion cells, support cells to ganglion cells, and optic nerve fibers.

In general, the present invention provides pharmaceutical compounds and compositions for treating or controlling in a subject an eye condition or disorder, which has an inflammatory component.

In one aspect, such an inflammatory component comprises a sequela of the eye condition or disorder.

In another aspect, such an inflammatory component comprises an etiology of the eye condition or disorder.

In yet another aspect, such a condition or disorder is selected from the group consisting of DR, AMD, DME, uveitis (including posterior uveitis, anterior uveitis, intermediate uveitis, panuveitis), corneal inflammatory diseases (such as corneal edema, exudation, or opacification), non-allergic conjunctivitis, keratoconjunctivitis, endophthalmitis, ocular neurodegeneration (including all types of glaucoma), optic neuritis, retinitis pigmentosa, and combinations thereof.

In still another aspect, a composition of the present invention comprises a modulator of PKC-θ, in an effective amount for treating or controlling such an eye condition or disorder.

In yet another aspect, a composition of the present invention comprises a compound that substantially inhibits, reduces, or interferes with, an activity of a cell signaling cascade involving PKC-θ, in an effective amount for treating or controlling such an eye condition or disorder.

In yet another aspect, a composition of the present invention comprises a PKC-θ inhibitor.

In still another aspect, such PKC-θ is a human PKC-θ.

In still another aspect, such PKC-θ is expressed in a cell or tissue associated with the human ocular system.

In yet another aspect, such PKC-θ is activated in a cell or tissue associated with the human ocular system.

In still another aspect, such a PKC-θ inhibitor is capable of down regulating a cell signaling pathway involving PKC-θ.

In yet another aspect, a composition of the present invention comprises a compound that is capable of inhibiting an activation of a human PKC-θ signaling pathway.

In a further aspect, a composition of the present invention comprises: (a) a PKC-θ inhibitor; and (b) an anti-inflammatory medicament.

In yet another aspect, the present invention provides a method for treating or controlling an eye condition or disorder of a subject. The method comprises administering a composition to an affected eye of the subject, which composition comprises a PKC-θ inhibitor; or a compound that is capable of inhibiting an activation of a human PKC-θ signaling pathway; or a combination thereof; in an effective amount for treating or controlling such eye condition or disorder.

In a further aspect, the present invention provides a method for treating or controlling degeneration of at least a component of an optic nerve system. The method comprises administering a composition to an affected eye, which composition comprises a PKC-θ inhibitor; or a compound that is capable of inhibiting an activation of a human PKC-θ signaling pathway; or a combination thereof; in an effective amount for treating or controlling such degeneration.

In still another aspect, such PKC-θ is over-expressed in a cell or tissue associated with the human optic nerve system.

In still another aspect, such PKC-θ is over-activated in a cell or tissue associated with the human optic nerve system.

Although an immune response is essential to initiate a repair of an affected site, an overactive or prolonged inflammatory response is damaging to otherwise healthy tissues surrounding the site of inflammation. For example, inflammation causes the blood vessels at the affected site to dilate to increase blood flow to the site. As a result, these dilated vessels become leaky. After prolonged inflammation, the leaky vessels can produce serious edema in, and impair the proper functioning of, the surrounding tissues (see; e.g., V. W. M. van Hinsbergh, Arteriosclerosis, Thrombosis, and Vascular Biology, Vol. 17, 1018 (1997)). In addition, a continued dominating presence of macrophages at the site of invasion continues the production of toxins (such as reactive oxygen species), inflammatory cytokines, and matrix-degrading enzymes (such as matrix metalloproteinases) by these cells, which are injurious to both the pathogen and the host's tissues. Therefore, an inappropriately vigorous activation of the immune system in response to non-infectious foreign materials should be controlled to limit the unintended damages to an otherwise healthy tissue.

Chronic stress imposed on cells, including those of the ocular system, resulting from the continued presence of high levels of inflammatory cytokines or toxins, can lead to the degeneration of these cells and, eventually, death if such stress is not relieved promptly. Such degeneration usually begins with disregulation of one or more biological compounds that are key to the maintenance of homeostasis.

The protein kinase C (“PKC”) family comprises twelve closely related isoforms of serine/threonine kinases. The PKC family is subdivided into three major groups. The conventional PKCs include the α, βI, βII, and γ isoforms and are activated by Ca²⁺ and diacylglycerol (“DAG”), a level of which can increase because of hyperglycemia or diabetic state. The novel PKCs include the δ, ε, η, and θ isoforms and are activated by DAG, but not Ca²⁺. The atypical PKCs include the ζ and λ/t isoforms, which are insensitive to both Ca²⁺ and DAG. PKC isoenzymes play a pivotal role as cellular mediators of signal transduction for hormones and growth factors as well as regulating diverse processes, including vascular haemodynamics, cellular proliferation and migration, neovascularization, enzyme activities, and gene expression of both growth factors and proto-oncogenes. Thus, amounts of PKC proteins are found in most types of tissues at regulated levels for normal body function. However, some PKC isozymes show distinctive distribution in different tissues. For example, PKC-βI and -βII are present in greatest amount in the brain and spleen. PKC-γ is seen mostly in the brain, and to a lesser degree, in the adrenal tissue. PKC-6 is concentrated in the brain, heart, spleen, lung, liver, ovary, pancreas, and adrenal tissues. PKC-ε is present in the brain, kidney, and pancreas. PKC-ζ; is found mostly in the brain, lung, and liver. PKC-θ is present predominantly in T lymphocytes and muscle cells.

There is growing evidence that PKC-θ selectively mediates the activation of transcription factors NF-κB (nuclear factor KB) and AP-1 (activator protein-1). K. Hayashi and A. Altman, Pharmacol. Rev., Vol. 55, 537 (2007). PKC-θ is expressed at relatively high levels in skeletal muscle, where it is suggested to play a role in signal transduction in both the developing and mature neuromuscular junction. Studies also suggest that PKC-θ is expressed in endothelial cells and involved in multiple processes essential for angiogenesis and wound healing, including the regulation of cell cycle progression and formation of capillary tubes. N. Meller et al., Stem Cells, Vol. 16, 178 (1998).

It is now known that NF-κB and AP-1 are key transcription factors that orchestrate expression of many genes involved in inflammation, angiogenesis, lymphoid differentiation, oncogenesis, and apoptosis. For example, activated NF-κB binds to and upregulates genes encoding proinflammatory cytokines, such as TNF-α, IL-1β, IL-2, IL-6, granulocyte-macrophage colony-stimulating factor (“GM-CSF”), macrophage colony-stimulating factor (“M-CSF”), and granulocyte colony-stimulating factor (“G-CSF”); chemokines, such as IL-8, macrophage inflammatory protein 1α (“MIP-1α”), and macrophage chemotactic protein 1 (“MCP-1”); inflammatory enzymes, such as inducible nitric oxide synthase (“iNOS”), cyclooxygenase-2 (“COX-2”), and 5-lipoxygenase; and adhesion molecules, such as intercellular adhesion molecule 1 (“ICAM-1”) and vascular-cell adhesion molecule 1 (“VCAM-1”). P. J. Barnes and M. Karin, New England J. Med., Vol. 336, No. 15, 1066 (1997). Adhesion molecules are facilitators of angiogenesis, which is the hallmark of back-of-the-eye diseases, such as DR and wet AMD.

Equally important, AP-1 has been shown to be a positive regulator of IL-8 and a critical upregulator of IL-2. IL-8 is a chemokine that potently recruits neutrophils to the site of inflammation, thus plays an important role in the initiation and maintenance of host inflammatory response. K. D. Ju et al., Ann. N.Y. Acad. Sci., Vol. 1090, 368 (2006). IL-2 is a potent cytokine for the activation and proliferation of T cells. M. Villalba and A. Altman, Curr. Cancer Drug Targets, Vol. 2, 125 (2002). It was shown recently that blockade of receptors for IL-2 with an IL-2 monoclonal antibody (daclizumab, Protein Design Laboratories, Inc.) resulted in remission of active uveitis. Z. Li et al., J. Immunol., Vol. 174, 5187 (2005). Moreover, NF-κB may be a co-stimulator of AP-1 in the upregulation of VEGF (S. Fujioka et al., Mol. Cell. Biol., Vol. 24, 7806 (2004)), which is ubiquitously found in proliferative conditions, including cancer, wet AMD, and DR. Thus, NF-κB can act synergistically with AP-1 to accelerate aberrant angiogenesis resulting from a diverse array of stimuli, which angiogenesis may not be as serious if each of the transcription factors acts alone.

The present inventors recognize that PKC-θ can play a central role in the pathogenesis of many specific conditions, disorders, or diseases of the eye, which have an inflammatory sequela, etiology, or both. However, such role of PKC-θ has not been found in, or suggested by, the prior art, explicitly or by inference.

Therefore, in one aspect, the present invention provides compositions and methods for treating or controlling an eye condition, disorder, or disease in a subject, which has an inflammatory component.

In another aspect, such compositions provide such treatment or control through inhibiting or antagonizing activity of human PKC-θ.

In still another aspect, such a condition, disorder, or disease is selected from the group consisting of DR, AMD, DME, uveitis (including posterior uveitis, anterior uveitis, intermediate uveitis, panuveitis), corneal inflammatory diseases (such as corneal edema, exudation, or opacification), non-allergic conjunctivitis, keratoconjunctivitis, ocular neurodegeneration (including all types of glaucoma), endophthalmitis, optic neuritis, retinitis pigmentosa, and combinations thereof.

In still another aspect, a composition of the present invention comprises a modulator of PKC-θ, in an effective amount for treating or controlling an eye condition, disorder, or disease selected from the group consisting of DR, AMD, DME, uveitis (including posterior uveitis, anterior uveitis, intermediate uveitis, panuveitis), corneal inflammatory diseases (such as corneal edema, exudation, or opacification), non-allergic conjunctivitis, keratoconjunctivitis, endophthalmitis, ocular neurodegeneration (including all types of glaucoma), optic neuritis, retinitis pigmentosa, and combinations thereof.

In yet another aspect, a composition of the present invention comprises a compound that substantially inhibits, reduces, or interferes with, an activity of a cell signaling cascade involving PKC-θ, in an effective amount for treating or controlling such a condition, disorder, or disease.

In a further aspect, a composition of the present invention comprises a compound that substantially inhibits, reduces, or interferes with, activation of PKC-θ, in an effective amount for treating or controlling such a condition, disorder, or disease.

In yet another aspect, a composition of the present invention comprises a PKC-θ inhibitor and an anti-inflammatory medicament. Preferably, such an anti-inflammatory medicament comprises a nonsteroidal compound.

The sequences of cDNA encoding human PKC-θ (SEQ. NO. 1) and of human PKC-θ protein (SEQ. NO. 2) are shown in the Sequence Listing below. The amino acid sequence between residues 380 and 634 of SEQ. NO. 2 represents the catalytic domain of PKC-θ, against which antibodies may be designed for inhibiting an activity of this enzyme. Design of antibodies against a protein having a known amino acid sequence or encoded by a known nuclei acid sequence can be readily accomplished.

In one aspect, a PKC-θ inhibitor comprises an agent which decreases the level of PKC-θ expression and/or activity. An agent that decreases the level of PKC-θ expression or activity can be one or more of: a PKC-θ antagonist (e.g., a PKC-θ binding protein that binds to PKC-θ but does not activate the enzyme); a PKC-θ nucleic acid molecule that can bind to a cellular PKC-θ nucleic acid sequence (e.g., mRNA) and inhibit expression of the protein (e.g., an antisense molecule or PKC-θ ribozyme); an antibody that specifically binds to PKC-θ protein (e.g., an antibody that disrupts PKC-θ's catalytic activity or an antibody that disrupts the ability of upstream activators to activate PKC-θ); an agent that decreases PKC-θ gene expression and/or activity (e.g., a small molecule that inhibits PKC-θ (e.g., rottlerin, disclosed below)).

In another aspect, PKC-θ expression is inhibited by decreasing the level of expression of an endogenous PKC-θ gene (e.g., by decreasing transcription of the PKC-θ gene). In one embodiment, transcription of the PKC-θ gene can be decreased by: altering the regulatory sequences of the endogenous PKC-θ gene (e.g., by the addition of a negative regulatory sequence, such as a DNA-biding site for a transcriptional repressor, or by the removal of a positive regulatory sequence, such as an enhancer or a DNA-binding site for a transcriptional activator).

### Small-Molecule PKC-θ Inhibitors

In another aspect, a PKC-θ inhibitor included in a composition of the present invention comprises rottlerin (also known as mallotoxin or 1-[6-[(3-acetyl-2,4,6-trihydroxy-5-methylphenyl)methyl]-5,7-dihydroxy-2,2-dimethyl-2H-1-benzopyran-8-yl]-3-phenyl-2-propen-1-one, available from Calbiochem, San Diego, Calif.) having Formula I, or a derivative or analogue thereof.

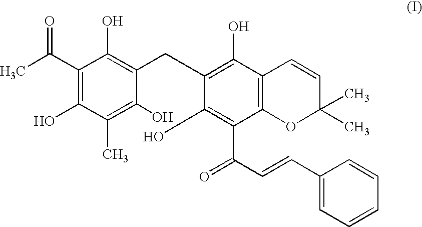

In another aspect, a PKC-θ inhibitor included in a composition of the present invention comprises a substituted diaminopyrimidine compound having Formula II, disclosed in U.S. Patent Application Publication US 2005/0222186 A1, which is incorporated herein by reference in its entirety.

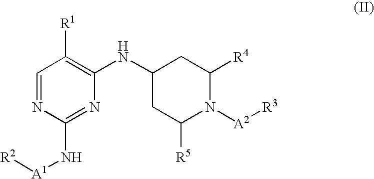

wherein R¹, R² and R³ are independently selected from the group consisting of substituted or unsubstituted phenyl, naphthyl, pyrrolyl, pyrazolyl, imidazolyl, 1,2,3-triazolyl, indolyl, benzimidazolyl, furanyl(furyl), benzofuranyl(benzofuryl), thiophenyl(thienyl), benzothiophenyl(benzothienyl), thiazolyl, isoxazolyl, pyridinyl, pyrimidinyl, quinolinyl and isoquinolinyl; R⁴ is hydrogen or methyl; R⁵ is hydrogen or methyl; A¹ is C₁₋₃ alkylene or ethyleneoxy (—CH₂—CH₂—O—); and A² is C₁₋₃ alkylene or ethyleneoxy (—CH₂—CH₂—O—); and hydrates, solvates, salts, or esters thereof.

Non-limiting examples of such compounds include [1-benzyl(4-piperidyl)]{2-[(2-pyridylmethyl)amino]-5-(3-thienyl)pyrimidin-4-yl}amine; {5-(4-methoxyphenyl)-2-[(4-pyridylmethyl)amino)pyrimidin-4-yl}[1-benzyl-(4-piperidyl)]amine; {5-phenyl-2-[(4-pyridylmethyl)amino)pyrimidin-4-yl}[1-benzyl(4-piperidy-1)]amine; {5-(4-chlorophenyl)-2-[(4-pyridylmethyl)amino)pyrimidin-4-yl}[1-benzyl(4-piperidyl)]amine; {5-(4-(N,N-dimethylamino)phenyl)-2-[(4-pyridylmethyl)amino)pyrimidin-4-yl}[1-benzyl(4-piperidyl)]amine; {5-(phenyl-4-carboxamido)-2-[(4-pyridylmethyl)amino)pyrimidin-4-yl}[1-benzyl(4-piperidyl)]-amine; {5-(4-carboxyphenyl)-2-[(4-pyridylmethyl)amino)pyrimidin-4-yl}[1-benzyl-(4-piperidyl)]amine; {5-(2-thienyl)-2-[(4-pyridylmethyl)amino)pyrimidin-4-yl}[1-benzyl(4-piperidyl)]amine; {5-(2-furanyl)-2-[(4-pyridylmethyl)amino)pyrimidin-4-yl}[1-benzyl(4-piperidyl)]amine; {5-(3-furanyl)-2-[(4-pyridylmethyl)amino)pyrimidin-4-yl}[1-benzyl(4-piperidyl)]amine; N(4)-(1-benzyl-piperidin-4-yl)-5-(3-chloro-4-fluoro-phenyl)-N(2)-pyridin-2-ylmethyl-pyrimidine-2,4-diamine; N-(3-[4-(1-benzyl-piperidin-4-ylamino)-2-[(pyridin-2-ylmethyl)-amino]-pyrimidin-5-yl}phenyl)-acetamide; 3-[4-(1-benzyl-piperidin-4-ylamino)-2-[(pyridin-2-ylmethyl)-amino]-pyrimidin-5-yl]-phenol; and 4-{4-(1-benzyl piperidin-4-ylamino)-2-[(pyridin-2-ylmethyl)-amino]-pyrimidin-5-yl}N,N-dimethyl-benzamide.

In another aspect, a PKC-θ inhibitor included in a composition of the present invention comprises a substituted pyridine compound having Formula III, disclosed in U.S. Patent Application Publication US 2006/0217417 A1, which is incorporated herein by reference in its entirety.

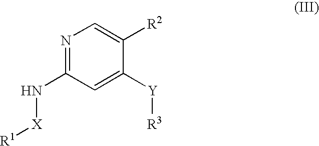

wherein X is a bond or C₁₋₆ substituted or unsubstituted alkyl wherein one or two of the methylene units can be replaced by an oxygen or sulfur atom; Y is —NH—, —O— or —S—; R¹ is a C₃₋₆ substituted or unsubstituted cycloalkyl, substituted or unsubstituted aryl or substituted or unsubstituted heteroaryl; R² is selected from the following group consisting of trifluoromethyl, cyano, —CONH₂, halogen, and nitro; and R³ is

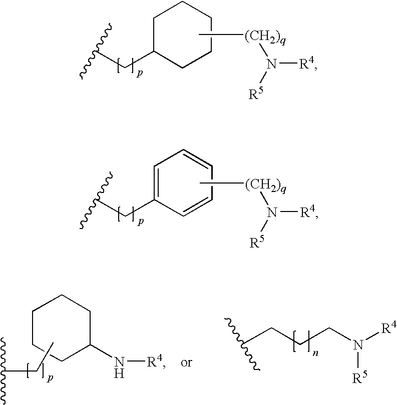

wherein p is an integer from 1 to 3, inclusive; q is an integer from 0 to 3, inclusive; n is an integer from 0 to 5, inclusive; R⁴ and R⁵ are each independently selected from the group consisting of hydrogen, C₁₋₆ substituted or unsubstituted alkyl, or wherein R⁴ and R⁵ together constitute methylene bridges which together with the nitrogen atom between them form a four to six-membered substituted or unsubstituted ring wherein one of the methylene groups is optionally replaced by an oxygen, sulfur or NR group, wherein R is hydrogen or C₁₋₆ substituted or unsubstituted alkyl; tautomers; and pharmaceutically acceptable salts, solvates or amino-protected derivatives thereof.

Non-limiting examples of the compounds having Formula III include 5-nitro-N4-piperidin-4-ylmethyl-N2-(2-trifluoromethoxy-benzyl)-pyridine-2,4-diamine; N2-(2,3-dichloro-benzyl)-5-nitro-N4-piperidin-4-ylmethyl-pyridine-2,4-diamine; N2-[2-(3-chloro-phenyl)-ethyl]-5-nitro-N4-piperidin-4-ylmethyl-pyridine-2,4-diamine; 5-nitro-N2-phenethyl-N4-piperidin-4-ylmethyl-pyridine-2,4-diamine; N4-(4-aminomethyl-cyclohexylmethyl)-5-nitro-N2-(2-trifluoromethoxy-benzyl)-pyridine-2,4-diamine; N4-(4-aminomethyl-cyclohexylmethyl)-N2-(2,3-dichloro-benzyl)-5-nitro-pyridine-2,4-diamine; N4-(4-aminomethyl-cyclohexylmethyl)-5-nitro-N2-phenethyl-pyridine-2,4-diamine; N4-(4-aminomethyl-cyclohexylmethyl)-N2-[2-(3-chloro-phenyl)-ethyl]-5-nitro-1-pyridine-2,4-diamine; N4-(4-aminomethyl-cyclohexylmethyl)-5-nitro-N2-(2-chloro-benzyl)-pyridine-2,4-diamine; N4-(4-trans-aminomethyl-cyclohexylmethyl)-5-nitro-N-2-(2-trifluoromethoxybenzyl)-pyridine-2,4-diamine; N4-(4-trans-amino-cyclohexylmethyl)-5-nitro-N2-(2-trifluoromethoxy-benzyl)-pyridine-2,4-diamine; 4-[(4-aminomethyl-cyclohexylmethyl)-amino]-6-(2-chloro-benzylamino)-nicotinamide; and 4-[(4-aminomethyl-cyclohexylmethyl)-amino]-6-(2-chloro-benzylamino)-nicotinonitrile.

In still another aspect, a PKC-θ inhibitor included in a composition of the present invention comprises a compound disclosed in U.S. Patent Application Publication US 2007/0142401 A1, which is incorporated herein by reference in its entirety. Non-limiting examples of such compounds include compounds having Formulae IV and V.

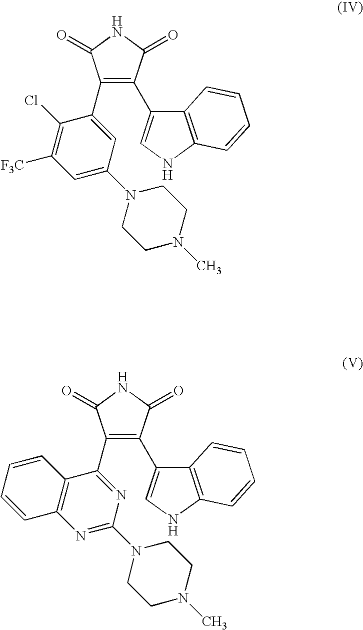

### Antibodies

In another aspect, a PKC-θ inhibitor included in a composition of the present invention comprises an antibody specifically reactive with human PKC-θ or a fragment of such antibody.

Anti-protein/anti-peptide antisera or monoclonal antibodies can be made by using standard protocols (See; e.g., “Antibodies: A Laboratory Manual” ed. by Harlow and Lane (Cold Spring Harbor Press, 1988)).

PKC-θ, or a portion or fragment thereof, can be used as an immunogen to generate antibodies that bind the component using standard techniques for polyclonal and monoclonal antibody preparation. The full-length protein can be used or, alternatively, antigenic peptide fragments of the protein can be used as immunogens.

Typically, a peptide is used to prepare antibodies by immunizing a suitable subject (e.g., rabbit, goat, mouse or other mammal) with the immunogen. An appropriate immunogenic preparation can contain, for example, a recombinant or a chemically synthesized PKC-θ peptide. See; e.g., U.S. Pat. Nos. 5,460,959; 5,994,127; 6,048,729; and 6,063,630, for exemplary methods of preparing the peptide; which patents are hereby expressly incorporated by reference in their entirety. The nucleotide and amino acid sequence of human PKC-θ is shown below (SEQ. NO.1). The preparation can further include an adjuvant, such as Freund's complete or incomplete adjuvant, or similar immunostimulatory agent. Immunization of a suitable subject with an immunogenic PKC-θ preparation induces a polyclonal anti-PKC antibody response.

Fragments of antibodies to PKC-θ, such as Fv, Fab, Fab′ and F(ab′)₂ fragments, can be generated by treating the antibody with an enzyme such as pepsin. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

In addition, antibodies produced by genetic engineering methods, such as chimeric or humanized monoclonal antibodies, comprising both human and non-human portions, which can be made using standard recombinant DNA techniques, can be used. Such chimeric or humanized monoclonal antibodies can be produced by genetic engineering using standard DNA techniques known in the art, for example using methods described in U.S. Pat. Nos. 5,721,108; 5,677,180 and 5,500,362 (Robinson et al.); U.S. Pat. No. 4,935,496 (Akira et al.); U.S. Pat. No. 5,807,715 (Morrison et al.); U.S. Pat. No. 4,816,567 (Cabilly et al.); which are incorporated herein by reference in their entirety; A. Y. Liu et al., PNAS, Vol. 84, No. 10, 3439 (1987); L. K. Sun et al., PNAS, Vol. 84, No. 1, 214 (1987); Y. Nishimura et al., Canc. Res., Vol. 47, 999 (1987).

In addition, a monoclonal antibody directed against human PKC-θ described herein can be made using standard techniques. For example, monoclonal antibodies can be generated in transgenic mice or in immune deficient mice engrafted with antibody-producing human cells. Methods of generating such mice are describe, for example, in S. L. Morrison et al., PNAS, Vol. 81, No. 21, 6851 (1984); N. Tuaillon et al., PNAS, Vol. 90, No. 8, 3720 (1993); U.S. Pat. No. 5,411,749. A human antibody-transgenic mouse or an immune deficient mouse engrafted with human antibody-producing cells or tissue can be immunized with human PKC-θ isoform described herein or an antigenic peptide thereof and splenocytes from these immunized mice can then be used to create hybridomas. Methods of hybridoma production are well known. Antibodies produced from such transgenic animal have been known to be well tolerated in human.

Monoclonal antibodies against human PKC-θ isoform disclosed herein can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., U.S. Pat. No. 5,969,108 (McCafferty et al.), which is incorporated herein by reference in its entirety, and A. D. Griffths et al., EMBO J, Vol. 12, No. 2, 725 (1993). In addition, a combinatorial library of antibody variable regions can be generated by mutating a known human antibody. For example, a variable region of a human antibody known to bind PKC-θ can be mutated by, for example, using randomly altered mutagenized oligonucleotides, to generate a library of mutated variable regions which can then be screened to bind to PKC-θ. Methods of inducing random mutagenesis within the CDR regions of immunoglobin heavy and/or light chains, methods of crossing randomized heavy and light chains to form pairings and screening methods can be found in, for example, U.S. Pat. No. 5,667,988 (Barbas III et al.), which is incorporated herein by reference in its entirety, and C. F. Barbas III et al., PNAS, Vol. 89, No. 10, 4457 (1992).

The immunoglobulin library can be expressed by a population of display packages, preferably derived from filamentous phage, to form an antibody display library. Non-limiting examples of methods and reagents particularly amenable for use in generating antibody display library can be found in, for example, U.S. Pat. No. 5,223,409 (Ladner et al.); U.S. Pat. No. 5,759,817 (Kang et al.); and U.S. Pat. No. 5,969,108 (McCafferty et al.); which are incorporated herein by reference in their entirety; A. D. Griffths et al. (1993) supra; H. Gram et al., PNAS, Vol. 89, No. 8, 3576 (1992); and C. F. Barbas III et al., PNAS, Vol. 88, No. 18, 7978 (1991). Once displayed on the surface of a display package (e.g., filamentous phage), the antibody library is screened to identify and isolate packages that express an antibody that binds PKC-θ described herein. In one embodiment, the primary screening of the library involves panning with an immobilized PKC-θ described herein and display packages expressing antibodies that bind the immobilized PKC-θ are selected.

In another aspect, antibodies to human PKC-θ may be obtained from Invitrogen, Carlsbad, Calif.; BD Biosciences Pharmigen, San Diego, Calif.; Novus Biologicals, Littleton, Colo.; or Epitomics, Inc., Burlingame, Calif.

### Antisense Nucleic Acid Sequences

Targeted inhibition of gene expression has become a fruitful approach for therapeutic intervention of diseases. Nucleic acid molecules that are “antisense” to a nucleotide sequence encoding human PKC-θ can be used as an agent that inhibits expression of human PKC-θ. An “antisense” nucleic acid includes a nucleotide sequence that is complementary to a “sense” nucleic acid encoding the protein, e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule that is complementary to the “coding region” of the coding strand of a nucleotide sequence encoding the protein can be used.

The nucleic acid sequence encoding human PKC-θ (SEQ. NO. 1) is shown in the Sequence Listing below. Given the coding sequence encoding the isozyme, an antisense nucleic acid can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

Non-limiting examples of antisense nucleic acids interfering with PKC-θ transcription include SEQ. NO. 3 (for V3 region), SEQ. NO. 4 and SEQ. NO. 5 (for C1 region); and SEQ. NO. 6 (for V1 region). Y. S. Niino et al., J. Biol. Chem., Vol. 276, No. 39, 36711 (2001). Other non-limiting examples of antisense nucleic acids (SEQ. NO. 7-13) are disclosed in U.S. Pat. No. 6,190,869; which is incorporated herein by reference in its entirety.

### RNA-Interfering PKC-θ Inhibitors

In another aspect, a PKC-θ inhibitor included in a composition of the present invention comprises a small nucleic acid molecule that downregulates, inhibits, or reduces the expression of PKC-θ, or the expression or activity of another gene involved in a pathway of PKC-θ gene expression. Non-limiting examples of such nucleic acid molecules include short interfering nucleic acid (“siNA”), short interfering RNA (“siRNA”), double stranded RNA (“dsRNA”), micro-RNA (“miRNA”), and short hairpin RNA (“shRNA”). Techniques for making these nucleic acid molecules are disclosed, for example, in U.S. Pat. Nos. 5,514,567; 5,561,222; 6,506,559; 7,022,828; 7,078,196; 7,176,304; 7,282,564; and 7,294,504; which are incorporated herein by reference in their entirety.

Alternatively, useful PKC-θ RNAis may be those available from Invitrogen, Carlsbad, Calif.

In yet another aspect, a PKC-θ inhibitor is included in a composition of the present invention in an amount from about 0.0001 to about 10 percent by weight of the composition. Alternatively, such PKC-θ inhibitor is present in a composition of the present invention in an amount from about 0.001 to about 5 percent (or from about 0.001 to about 2, or from about 0.001 to about 1, or from about 0.001 to about 0.5, or from about 0.001 to about 0.2, or from about 0.001 to about 0.1, or from about 0.01 to about 0.1, or from about 0.01 to about 0.5, or from about 0.01 to about 1, or from about 0.001 to about 0.01, or from about 0.001 to about 0.1 percent, or from about 0.1 to about 5, or from about 0.1 to about 2, or from about 0.1 to about 1, or from about 0.1 to about 0.5, or from about 0.1 to about 0.2) by weight of the composition.

In another aspect, a composition of the present invention comprises: (a) a PKC-θ inhibitor; and (b) an anti-inflammatory agent.

In one embodiment, such an anti-inflammatory agent is selected from the group consisting of non-steroidal anti-inflammatory drugs (“NSAIDs”), dissociated glucocorticoid receptor agonists (“DIGRAs”), peroxisome proliferator-activated receptor (“PPAR”) ligands (including PPAR antagonists), combinations thereof, and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

In another aspect, the anti-inflammatory agent is a dissociated glucocorticoid receptor agonist (“DIGRA”) is a compound that is capable of binding to the glucocorticoid receptor (which is a polypeptide) and, upon binding, is capable of producing differentiated levels of transrepression and transactivation of gene expression. Thus, in one aspect, DIGRAs have the anti-inflammatory property similar to glucocorticosteroids but with lower levels of side effects of glucocorticosteroids. In another aspect, the DIGRA comprises a non-steroidal compound.

A useful DIGRA for a composition of the present invention can be any one of the compounds disclosed in U.S. Patent Application Publications 2004/0029932, 2004/0162321, 2004/0224992, 2005/0059714, 2005/0176706, 2005/0203128, 2005/0234091, 2005/0282881, 2006/0014787, 2006/0030561, 2006/0116396, 2006/0189646, 2006/0189647, and 2008/0009437, all of which are incorporated herein by reference in their entirety.

In certain embodiments, a DIGRA included in some compositions of the present invention has Formula VI or VII.

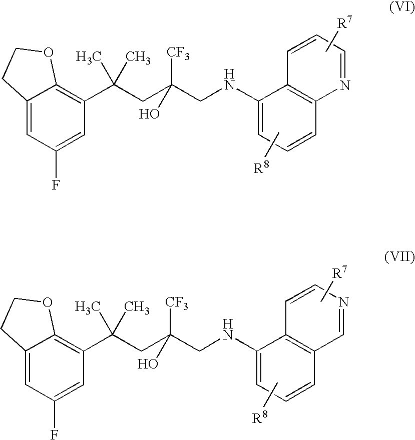

wherein R⁷ and R⁸ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In still another embodiment, said DIGRA has Formula VIII.

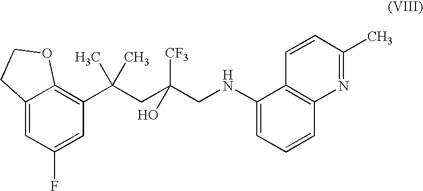

In another aspect of the present invention, an anti-inflammatory agent is a PPAR-binding molecule. In one embodiment, such a PPAR-binding molecule is a PPARα-, PPARδ-, or PPARγ-binding molecule. In another embodiment, such a PPAR-binding molecule is a PPARα, PPARδ, or PPARγ agonist. Such a PPAR ligand binds to and activates PPAR to modulate the expression of genes containing the appropriate peroxisome proliferator response element in its promoter region.

PPARγ agonists can inhibit the production of TNF-α and other inflammatory cytokines by human macrophages (C-Y. Jiang et al., Nature, Vol. 391, 82-86 (1998)) and T lymphocytes (A. E. Giorgini et al., Horm. Metab. Res. Vol. 31, 1-4 (1999)). More recently, the natural PPARγ agonist 15-deoxy-Δ-12,14-prostaglandin J2 (or “15-deoxy-Δ-12,14-PG J2”), has been shown to inhibit neovascularization and angiogenesis (X. Xin et al., J. Biol. Chem. Vol. 274:9116-9121 (1999)) in the rat cornea. Spiegelman et al., in U.S. Pat. No. 6,242,196 (incorporated herein by reference in its entirety) disclose methods for inhibiting proliferation of PPARγ-responsive hyperproliferative cells by using PPARγ agonists; numerous synthetic PPARγ agonists are disclosed by Spiegelman et al., as well as methods for diagnosing PPARγ-responsive hyperproliferative cells. All documents referred to herein are incorporated by reference. PPARs are differentially expressed in diseased versus normal cells. PPARγ is expressed to different degrees in the various tissues of the eye, such as some layers of the retina and the cornea, the choriocapillaris, uveal tract, conjunctival epidermis, and intraocular muscles (see, e.g., U.S. Pat. No. 6,316,465).

In one aspect, a PPARγ agonist used in a composition or a method of the present invention is a thiazolidinedione, a derivative thereof, or an analog thereof. Non-limiting examples of thiazolidinedione-based PPARγ agonists include pioglitazone, troglitazone, ciglitazone, englitazone, rosiglitazone, and chemical derivatives thereof. Other PPARγ agonists include Clofibrate (ethyl 2-(4-chlorophenoxy)-2-methylpropionate), clofibric acid (2-(4-chlorophenoxy)-2-methylpropanoic acid), GW 1929 (N-(2-benzoylphenyl)-O-{2-(methyl-2-pyridinylamino)ethyl}-L-tyrosine), GW 7647 (2-{{4-{2-{{(cyclohexylamino)carbonyl}(4-cyclohexylbutyl)amino}ethyl}phenyl}thio}-2-methylpropanoic acid), and WY 14643 ({{4-chloro-6-{(2,3-dimethylphenyl)amino}-2-pyrimidinyl}thio}acetic acid). GW 1929, GW 7647, and WY 14643 are commercially available, for example, from Koma Biotechnology, Inc. (Seoul, Korea). In one embodiment, the PPARγ agonist is 15-deoxy-Δ-12,14-PG J2.

Non-limiting examples of PPAR-α agonists include the fibrates, such as fenofibrate and gemfibrozil. A non-limiting example of PPAR-δ agonist is GW501516 (available from Axxora LLC, San Diego, Calif. or EMD Biosciences, Inc., San Diego, Calif.).

In still another aspect of the present invention, the anti-inflammatory agent may be an anti-angiogenic agent. Such an agent can be a VEGF inhibitor, such as Lucentis® or Avastin®. VEGF has been known to act either directly or indirectly as a potent pro-inflammatory cytokine. For example, overexpression of VEGF results in leukocyte adhesion at the leading edge of pathological neovascularization. S. Ishida et al., “VEGF₁₆₄-mediated inflammation is required for pathological, but not physiological, ischemia-induced retinal neovascularization,” J. Exp. Med., Vol. 198, No. 3, 483 (2003). VEGF can also augment pro-inflammatory T cell differentiation. F. Mor et al., “Angiogenesis-inflammation cross-talk: vascular endothelial growth factor is secreted by activated T cells and induces Th₁polarization,” J. Immunol., Vol. 172, 4618 (2004).

Each of said anti-inflammatory agents, when included in a composition, is present in a composition of the present invention in an amount from about 0.001 to about 5 percent (or from about 0.001 to about 2, or from about 0.001 to about 1, or from about 0.001 to about 0.5, or from about 0.001 to about 0.2, or from about 0.001 to about 0.1, or from about 0.01 to about 0.1, or from about 0.01 to about 0.5, or from about 0.001 to about 0.01, or from about 0.001 to about 0.1 percent) by weight of the composition.

### Other Suitable Ingredients in a Composition of the Present Invention

In one aspect, in addition to a PKC-θ inhibitor, a composition of the present invention comprises a liquid medium. In one embodiment, the liquid medium comprises an aqueous solution. In another aspect, the liquid medium comprises a non-aqueous formulation.

In another aspect, a composition of the present invention further comprises a material selected from the group consisting of preservatives, antimicrobial agents, surfactants, buffers, tonicity-modifying agents, chelating agents, viscosity-modifying agents, co-solvents, oils, humectants, emollients, stabilizers, antioxidants and combinations thereof.

Water-soluble preservatives that may be employed in a composition of the present invention include benzalkonium chloride, benzoic acid, benzoyl chloride, benzyl alcohol, chlorobutanol, calcium ascorbate, ethyl alcohol, potassium sulfite, sodium ascorbate, sodium benzoate, sodium bisulfite, sodium bisulfate, sodium thiosulfate, thimerosal, methylparaben, ethylparaben, propylparaben, polyvinyl alcohol, phenylethyl alcohol, quaternary alkyl ammonium salts (such as Polyquatemium-1 or Polyquaternium-10), hydrogen peroxide, and urea peroxide, and biguanides. Other preservatives useful in the present invention include, but are not limited to, the FDA-approved preservative systems for food, cosmetics, and pharmaceutical preparations. These agents may be present in individual amounts of from about 0.001 to about 5 percent by weight (preferably, from about 0.001 percent to about 2 percent by weight; more preferably, from about 0.001 percent to about 1 percent by weight, or from about 0.001 percent to about 0.1 percent by weight).

In one embodiment, a composition of the present invention comprises an anti-microbial agent. Non-limiting examples of antimicrobial agents include the quaternary ammonium compounds and bisbiguanides. Representative examples of quaternary ammonium compounds include benzalkonium halides and balanced mixtures of n-alkyl dimethyl benzyl ammonium chlorides. Other examples of antimicrobial agents include polymeric quaternary ammonium salts used in ophthalmic applications such as poly[(dimethyliminio)-2-butene-1,4-diyl chloride], [4-tris(2-hydroxyethyl)ammonio]-2-butenyl-w-[tris(2-hydroxyethyl)ammonio]dichloride (chemical registry number 75345-27-6) generally available as Polyquatemium-1® from ONYX Corporation.

Non-limiting examples of antimicrobial biguanides include the bis(biguanides), such as alexidine or chlorhexidine or salts thereof, and polymeric biguanides such as polymeric hexamethylene biguanides (“PHMB”) and their water-soluble salts, which are available, for example, from Zeneca, Wilmington, Del.

In one aspect, a composition of the present invention includes a disinfecting amount of an antimicrobial agent that will at least prevent the growth of microorganisms in the formulations employed. Preferably, a disinfecting amount is that which will reduce the microbial burden by two log orders in four hours and more preferably by one log order in one hour. Typically, such agents are present in concentrations ranging from about 0.00001 to about 0.5 percent (w/v); preferably, from about 0.00003 to about 0.5 percent (w/v); and more preferably, from about 0.0003 to about 0.1 percent (w/v).

In another aspect, a composition of the present invention comprises a surfactant. Suitable surfactants can be amphoteric, cationic, anionic, or non-ionic, which may be present (individually or in combination) in amounts up to 15 percent, preferably up to 5 percent weight by volume (w/v) of the total composition (solution). In one embodiment, the surfactant is an amphoteric or non-ionic surfactant, which when used imparts cleaning and conditioning properties. The surfactant should be soluble in the lens care solution and non-irritating to eye tissues. Many non-ionic surfactants comprise one or more chains or polymeric components having oxyalkylene (—O—R—) repeating units wherein R has 2 to 6 carbon atoms. Preferred non-ionic surfactants comprise block polymers of two or more different kinds of oxyalkylene repeat units. Satisfactory non-ionic surfactants include polyethylene glycol esters of fatty acids, polysorbates, polyoxyethylene, or polyoxypropylene ethers of higher alkanes (C₁₂-C₁₈). Non-limiting examples of the preferred class include polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006). The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

Various other ionic as well as amphoteric and anionic surfactants suitable for use in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Amphoteric surfactants suitable for use in a composition according to the present invention include materials of the type offered commercially under the trade name “Miranol.” Another useful class of amphoteric surfactants is exemplified by cocoamidopropyl betaine, commercially available from various sources.

The foregoing surfactants will generally be present in a total amount from 0.001 to 5 percent weight by volume (w/v), or 0.01 to 5 percent, or 0.01 to 2 percent, or 0.1 to 1.5 percent (w/v).

In another aspect, the pH of a composition of the present invention is maintained within the range of 5 to 8, preferably about 6 to 8, more preferably about 6.5 to 7.8. Non-limiting examples of suitable buffers include boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred, particularly for enhancing the efficacy of biguanides, when they are used in compositions of the present invention. Generally, buffers will be used in amounts ranging from about 0.05 to 2.5 percent by weight, and preferably, from 0.1 to 1.5 percent. In certain embodiments of this invention, the compositions comprise a borate or mixed phosphate buffer, containing one or more of boric acid, sodium borate, potassium tetraborate, potassium metaborate, or mixtures of the same.

In addition to buffering agents, in some instances it may be desirable to include chelating or sequestering agents in the present compositions in order to bind metal ions, which might otherwise react with the lens and/or protein deposits and collect on the lens. Ethylene-diaminetetraacetic acid (“EDTA”) and its salts (disodium) are preferred examples. They are usually added in amounts ranging from about 0.01 to about 0.3 weight percent. Other suitable sequestering agents include phosphonic acids, gluconic acid, citric acid, tartaric acid, and their salts; e.g., sodium salts.

In another aspect, compositions of the present invention comprise a tonicity-adjusting agent, to approximate the osmotic pressure of normal lacrimal fluid, which is equivalent to a 0.9 percent solution of sodium chloride or 2.5 percent of glycerol solution. Non-limiting examples of suitable tonicity-adjusting agents include, but are not limited to, sodium and potassium chloride, calcium and magnesium chloride, dextrose, glycerin, mannitol, and sorbitol. These agents are typically used individually in amounts ranging from about 0.01 to 2.5 percent (w/v) and preferably, form about 0.2 to about 1.5 percent (w/v). Preferably, the tonicity-adjusting agent will be employed in an amount to provide a final osmotic value of 200 to 450 mOsm/kg; more preferably, between about 250 to about 350 mOsm/kg, and most preferably between about 280 to about 320 mOsm/Kg.

In another aspect, it may be desirable to include one or more water-soluble viscosity-modifying agents in the compositions of the present invention. Because of their demulcent effect, viscosity-modifying agents have a tendency to enhance the patient's comfort by means of a lubricating film on the eye. Included among the water-soluble viscosity-modifying agents are the cellulose polymers like hydroxyethyl or hydroxypropyl cellulose, carboxymethyl cellulose and the like. Such viscosity-modifying agents may be employed in amounts ranging from about 0.01 to about 4 weight percent or less. The present compositions may also include optional demulcents.

In addition, a composition of the present invention can include additives such as co-solvents, oils, humectants, emollients, stabilizers, or antioxidants for a variety of purposes. These additives may be present in amounts sufficient to provide the desired effects, without impacting the performance of other ingredients.

### Methods of Administration

In embodiments wherein a PKC-θ inhibitor comprises a nucleic acid molecule, such molecule can be administered into a subject using in vivo gene therapy techniques (such as those disclosed in U.S. Pat. No. 5,399,346; which is incorporated herein by reference in its entirety). The nucleic-acid PKC-θ inhibitor incorporated into cells (e.g., ocular cells) of the host suppresses the in vivo activity of PKC-θ and produces neuroprotection to the subject.

For in vivo administration, the cells can be in the subject and the nucleic acid can be administered in a pharmaceutically acceptable carrier. The subject can be any animal in which it is desirable to selectively express a nucleic acid in a cell. In a preferred embodiment, the animal of the present invention is a human. In addition, non-human animals which can be treated by a method of this invention can include, but are not limited to, non-human primates, cats, dogs, birds, horses, cows, goats, sheep, guinea pigs, hamsters, gerbils and rabbits, as well as any other animal in which selective expression of a nucleic acid in a cell can be carried out according to the methods described herein.

In the method described above, which includes the introduction of an exogenous nucleic acid into the cells of a subject (i.e., gene transduction or transfection), a nucleic acid of the present invention can be in the form of naked DNA or RNA or the nucleic acids can be in a vector for delivering the nucleic acid to the cells for expression of the nucleic acid inside the cell. The vector can be a commercially available preparation, such as those disclosed herein above. Delivery of the nucleic acid or vector to cells can be via a variety of mechanisms. As one example, delivery can be via a liposome, using commercially available liposome preparations such as Lipofectin®, Lipofectamine® (GIBCO-BRL, Inc., Gaithersburg, Md.), Superfect® (Qiagen Inc., Valencia, Calif.) and Transfectam® (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a sonoporation machine (ImaRx Pharmaceutical Corp., Tucson, Ariz.).

As one example, vector delivery can be via a viral system, such as a retroviral vector system which can package a recombinant retroviral genome. The recombinant retrovirus can then be used to infect and thereby deliver nucleic acid to the infected cells. The exact method of introducing the nucleic acid into mammalian cells is, of course, not limited to the use of retroviral vectors. Other techniques are widely available for this procedure including the use of adenoviral vectors, adeno-associated viral (“AAV”) vectors, lentiviral vectors, pseudotyped retroviral vectors, and pox virus vectors, such as vaccinia virus vectors. Physical transduction techniques can also be used, such as liposome delivery and receptor-mediated and other endocytosis mechanism. A method disclosed herein can be used in conjunction with any of these or other commonly used gene transfer methods.

The nucleic acid and the nucleic acid delivery vehicles of this invention, (e.g., viruses; liposomes, plasmids, vectors) can be in a pharmaceutically acceptable carrier for in vivo administration to a subject. By “pharmaceutically acceptable” is meant a material that is not biologically or otherwise undesirable, i.e., the material may be administered to a subject, along with the nucleic acid or vehicle, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The nucleic acid or vehicle may be administered orally, parenterally (e.g., intravenously), by intramuscular injection, by intraperitoneal injection, transdermally, extracorporeally, topically or the like. The exact amount of the nucleic acid or vector required will vary from subject to subject, depending on the species, age, weight and general condition of the subject, the severity or mechanism of any disorder being treated, the particular nucleic acid or vehicle used, its mode of administration and the like.

The compounds of this invention (e.g., nucleic acids, proteins, polypeptides, or small molecules) can be administered to a cell of a subject either in vivo or ex vivo. For administration to a cell of the subject in vivo, as well as for administration to the subject, the compounds of this invention can be administered orally, parenterally (e.g., intravenously), by intramuscular injection, by intraperitoneal injection, subcutaneous injection, transdermally, extracorporeally, topically, mucosally or the like.

Depending on the intended mode of administration, the compounds of the present invention can be in pharmaceutical compositions in the form of solid, semi-solid or liquid dosage forms, such as, for example, tablets, pills, capsules, powders, liquids, suspensions, lotions, creams, gels, or the like. The compositions will include, as noted above, an effective amount of the selected compound, possibly in combination with a pharmaceutically acceptable carrier and, in addition, may include other medicinal agents, pharmaceutical agents, carriers, adjuvants, diluents, etc., many of which are disclosed herein.

Parenteral administration of the compounds of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, subcutaneous, intramuscular, intraperitoneal, intravenous and intratracheal routes. Parenteral administration can involve use of a slow release or sustained release system such that a constant dosage is maintained. See, e.g., U.S. Pat. No. 3,610,795, which is incorporated by reference herein. These compounds can be present in a pharmaceutically acceptable carrier, which can also include a suitable adjuvant.

The exact amount of the compound required will vary from subject to subject, depending on the species, age, weight and general condition of the subject, the particular compound used, its mode of administration and the like. An appropriate amount can be determined by one of ordinary skill in the art using only routine experimentation given the teachings herein.

If ex vivo methods are employed, cells or tissues can be removed and maintained outside the subject's body according to standard protocols well known in the art. The compounds of this invention can be introduced into the cells via known mechanisms for uptake of small molecules into cells (e.g., phagocytosis, pulsing onto class I MHC-expressing cells, liposomes, etc.). The compounds of this invention can also be linked to the homeodomain of Antennapedia for introduction, i.e. internalization of the compound, into cells (P. Prochiantz, Curr. Opin. Neurobiol., Vol. 6, No. 5, 629 (1996)). The cells can then be infused (e.g., in a pharmaceutically acceptable carrier) or transplanted back into the subject per standard methods for the cell or tissue type. Standard methods are known for transplantation or infusion of various cells into a subject.

The following examples serve to illustrate some non-limiting compositions of the present invention. The ingredients shown in each of Tables 1-10 are mixed to form a pharmaceutical composition for treating or controlling ocular neurodegenerative conditions. The composition may be further sterilized prior to administration to the subject, according to known methods of sterilization of pharmaceutical compositions.

### EXAMPLE 1

TABLE 1

| | Amount |
|---|---|
| Ingredient | (percent w/v) |
| Polyhexamethylenebiguanide HCl (as a 20 percent | 0.00047 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Poloxamine (Tetronic ® 1107 from BASF) | 1.00 |
| Tetrasodium etidronate (as a 30 percent w/w solution, | 0.01 |
| available under the mark DeQuest ® 2016 | |
| from Monsanto Co.) | |
| Rottlerin | 0.02 |
| Hydrochloric acid (1N) or sodium hydroxide (1N) | as required to |
| | adjust pH to 7-7.5 |
| Purified water | q.s. to 100 |

### EXAMPLE 2

TABLE 2

| | Amount |
|---|---|
| Ingredient | (percent w/v) |
| Polyhexamethylenebiguanide HCl (as a 20 percent | 0.00047 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Compound having Formula IV | 0.05 |
| Hydrochloric acid (1N) or sodium hydroxide (1N) | as required to |
| | adjust pH to 7-7.5 |
| Purified water | q.s. to 100 |

### EXAMPLE 3

TABLE 3

| | Amount |
|---|---|
| Ingredient | (percent w/v) |
| Polyhexamethylenebiguanide HCl (as a 20 percent w/w | 0.0006 |
| solution available under the mark Cosmocil CQ, from ICI | |
| Chemical Co.) | |
| Phosphate buffer | 0.5 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 | |
| from Monsanto Co.) | |
| Compound having Formula V | 0.06 |
| Purified water | q.s. to 100 |

### EXAMPLE 4

TABLE 4

| | Amount |
|---|---|
| Ingredient | (percent w/v) |
| Polyhexamethylenebiguanide HCl (as a 20 percent | 0.0005 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| PKC-θ Antibody catalog No. 610089 from BD | 0.05 |
| Biosciences Pharmigen, San Diego, California | |
| Hydrochloric acid (1N) or sodium hydroxide (1N) | as required to |
| | adjust pH to 7-7.5 |
| Purified water | q.s. to 100 |

### EXAMPLE 5

TABLE 5

| | Amount |
|---|---|
| Ingredient | (percent w/v) |
| Polyhexamethylenebiguanide HCl (as a 20 percent | 0.0005 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 | |
| from Monsanto Co.) | |
| Anti-human PKC-θ monoclonal antibody 2040-1 | 0.03 |
| humanized (2040-1 is a product of Epitomics, Inc., | |
| Burlingame, California) | |
| Hydrochloric acid (1N) or sodium hydroxide (1N) | as required to |
| | adjust pH to 7-7.5 |
| Purified water | q.s. to 100 |

### EXAMPLE 6

TABLE 6

| | Amount |
|---|---|
| Ingredient | (percent w/v) |
| Polyhexamethylenebiguanide HCl (as a 20 percent | 0.00047 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 | |
| from Monsanto Co.) | |
| Chloroquine | 0.001 |
| Antisense nucleotide having SEQ. NO. 6 | 0.01 |
| Hydrochloric acid (1N) or sodium hydroxide (1N) | as required to |
| | adjust pH to 7-7.5 |
| Purified water | q.s. to 100 |

### EXAMPLE 7

TABLE 7

| | Amount |
|---|---|
| Ingredient | (percent w/v) |
| Polyhexamethylenebiguanide HCl (as a 20 percent | 0.0005 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| RNAi catalog No. 1299003, Invitrogen, Carlsbad, | 0.03 |
| California | |
| Hydrochloric acid (1N) or sodium hydroxide (1N) | as required to |
| | adjust pH to 7-7.5 |
| Purified water | q.s. to 100 |

### EXAMPLE 8

TABLE 8

| | Amount |
|---|---|
| Ingredient | (percent w/v) |
| Polyhexamethylenebiguanide HCl (as a 20 percent | 0.0005 |
| w/w solution available under the mark Cosmocil CQ, | |
| from ICI Chemical Co.) | |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 | |
| from Monsanto Co.) | |
| Antisense nucleotide having SEQ. NO. 13 | 0.02 |
| Hydrochloric acid (1N) or sodium hydroxide (1N) | as required to |
| | adjust pH to 7-7.5 |
| Purified water | q.s. to 100 |

### EXAMPLE 9

TABLE 9

| | Amount |
|---|---|
| Ingredient | (percent w/v) |
| Urea peroxide | 0.06 |
| Boric acid | 0.64 |
| Sodium borate | 0.12 |
| Edetate disodium | 0.11 |
| Sodium chloride | 0.49 |
| Polysorbate 80 | 0.7 |
| Tetrasodium etidronate (as a 30 percent w/w solution, | 0.01 |
| available under the mark DeQuest ® 2016 | |
| from Monsanto Co.) | |
| [1-benzyl(4-piperidyl)]{2-[(2-pyridylmethyl)amino]-5- | 0.02 |
| (3-thienyl)pyrimidin-4-yl}amine | |
| Hydrochloric acid (1N) or sodium hydroxide (1N) | as required to |
| | adjust pH to 7-7.5 |
| Purified water | q.s. to 100 |

### EXAMPLE 10

TABLE 10

| | Amount |
|---|---|
| Ingredient | (percent w/v) |
| Polyquaternium-1 | 0.005 |
| Boric acid | 0.64 |
| Sodium borate | 0.12 |
| Edetate disodium | 0.11 |
| Sodium chloride | 0.49 |
| Poloxamine (Tetronic ® 1107 from BASF) | 1.00 |
| 4-[(4-aminomethyl-cyclohexylmethyl)-amino]-6- | 0.05 |
| (2-chloro-benzylamino)-nicotinamide | |
| Cyclosporine | 0.05 |
| Hydrochloric acid (1N) or sodium hydroxide (1N) | as required to |
| | adjust pH to 7-7.5 |
| Purified water | q.s. to 100 |

In another aspect, a preservative other than polyhexamethylenebiguanide HCl may be used in any one of the foregoing formulation, in a suitably effective amount.

In still another aspect, a composition can be free of preservative if it is formulated to be used as a unit-dose composition. In such a case, the composition is packaged in an individual container that is opened and the contents of the container are used only once.

The present invention also provides a method for treating or controlling an eye condition or disorder having an inflammatory component. The method comprises applying a composition to an affected eye, wherein the composition comprises compound that substantially inhibits, reduces, or interferes with, an activity of a cell signaling cascade involving PKC-θ, antagonizes PKC-θ, or inhibits the activation of PKC-θ, or a combination thereof, in an effective amount for treating or controlling such eye condition or disorder.

In one aspect, such a condition or disorder is selected from the group consisting of DR, AMD (including dry and wet AMD), DME, uveitis (including posterior uveitis, anterior uveitis, intermediate uveitis, panuveitis), corneal inflammatory diseases (such as corneal edema, exudation, or opacification), non-allergic conjunctivitis (such as viral, bacterial, chlamydial, and giant papillary conjunctivitis), keratoconjunctivitis, endophthalmitis, ocular neurodegeneration (including all types of glaucoma), optic neuritis, retinitis pigmentosa, and combinations thereof.

In one aspect, one or more PKC-θ inhibitors are incorporated into a formulation for topical administration, systemic administration, periocular injection, or intravitreal injection. A formulation can desirably comprise a carrier that provides a sustained release of the active ingredients, such as for a period longer than about 1 week (or longer than about 1, 2, 3, 4, 5, or 6 months). In certain embodiments, the sustained-release formulation desirably comprises a carrier that is insoluble or only sparingly soluble in the ocular environment. Such a carrier can be an oil-based liquid, emulsion, gel, or semisolid. Non-limiting examples of oil-based liquids include castor oil, peanut oil, olive oil, coconut oil, sesame oil, cottonseed oil, corn oil, sunflower oil, fish-liver oil, arachis oil, and liquid paraffin.

In one embodiment, a composition of the present invention can be injected intravitreally to control an eye condition or disorder having an inflammatory component, or a progression of an ocular neurodegenerative disease, using a fine-gauge needle, such as 25-33 gauge. Typically, an amount from about 25 μl to about 100 μl of a composition comprising one or more PKC-θ inhibitors is administered into a patient. A concentration of such a PKC-θ inhibitor or combination thereof is selected from the ranges disclosed above.

In another aspect, one or more PKC-θ inhibitors is incorporated into an ophthalmic device or system that comprises a biodegradable material, and the device is implanted into the posterior cavity of a diseased eye to provide a long-term (e.g., longer than about 1 week, or longer than about 1, 2, 3, 4, 5, or 6 months) control an eye condition or disorder having an inflammatory component, or a progression of an ocular neurodegenerative disease. In one aspect, such control is achieved by reducing the levels of pro-inflammatory cytokines in one or more tissues of the eye over a long period of time.

In still another aspect, a method for controlling an eye condition or disorder having an inflammatory component, or a progression of an ocular neurodegenerative disease comprises: (a) providing a composition comprising one or more PKC-θ inhibitors; and (b) administering to a subject an effective amount of the composition at a frequency sufficient to control the eye condition or progression of the ocular degenerative disease.

In one embodiment, one or more PKC-θ inhibitors are selected from among those disclosed above.

In still another embodiment, the present invention provides a method for controlling progression of optic nerve degeneration in a subject having hypertensive glaucoma. The method comprises: (a) administering a composition comprising one or more PKC-θ inhibitors to an eye of said subject; and (b) administering to the subject an intraocular-pressure (“IOP”) lowering drug, wherein the composition and the IOP lowering drug are administered in effective amounts at a frequency sufficient to control the progression of optic nerve degeneration. Non-limiting examples of IOP lowering drugs include prostaglandin analogs (lantanoprost, travoprost, bimatoprost), β-receptor antagonists (timolol maleate), α₂-adrenegic agonists (brionidine, clonidine), carbonic anhydrases (dorzolamide, brinzolamide), cholinomimetics (pilocarpine, carbachol), and inhibitors of acetylcholinesterase such as echothiophate (phospholine iodide).

In preferred embodiment, a composition of the present invention is administered intravitreally. In still another aspect, a composition of the present invention is incorporated into an ophthalmic implant system or device, and the implant system or device is surgically implanted in the vitreous cavity of the patient for the sustained or long-term release of the active ingredient or ingredients. A typical implant system or device suitable for use in a method of the present invention comprises a biodegradable matrix with the active ingredient or ingredients impregnated or dispersed therein. Non-limiting examples of ophthalmic implant systems or devices for the sustained-release of an active ingredient are disclosed in U.S. Pat. Nos. 5,378,475; 5,773,019; 5,902,598; 6,001,386; 6,051,576; and 6,726,918; which are incorporated herein by reference.

In yet another aspect, a composition of the present invention is injected into the vitreous once a month, or once every two, three, four, five, or six months. In another aspect, the composition is implanted in the patient and is replaced at a frequency of, for example, once a year or at a suitable frequency that is determined to be appropriate for controlling the progression of the ocular degenerative disease.

### Combination Therapy

Ocular inflammation can affect IOP by either directly or indirectly altering aqueous humor dynamics. Uveitis involving both the anterior (iris and ciliary body) and posterior uvea (choroid) is the inflammation most frequently associated with glaucoma. Acute inflammatory glaucoma with an open anterior chamber angle usually results from direct obstruction of aqueous outflow channels by platelets, immune system cells, whose aggregation is favored by increased aqueous protein and high concentrations within the trabecular meshwork. Moreover, many mediators of inflammation, including cytokines, prostaglandins, leukotrienes, platelet activating factor, and reactive oxide species, can also affect IOP. And it is known that prolonged increased IOP leads to ocular neurodegeneration.

Thus, a composition or a method of the present invention can be used in conjunction with other therapeutic, adjuvant, or prophylactic agents or methods commonly used to control (a) an increase of intraocular pressure, (b) a loss of neuronal cells of the retinal layers (such as retinal ganglion cells, Müller cells, amacrine cells, bipolar cells, horizontal cells, and photoreceptors) or (c) both, thus providing an enhanced overall treatment or enhancing the effects of the other therapeutic, prophylactic, or adjunctive agents or methods used to treat and manage the different ocular neurodegenerative diseases.

High doses may be required for some currently used therapeutic agents, or high frequency for currently used methods, to achieve levels to effectuate the target response, but may often be associated with a greater frequency of adverse effects. Thus, combined use of a composition of the present invention, with agents or methods commonly used to control progression of ocular nerve damage allows the use of relatively lower doses of such other agents, or frequency of such other methods, resulting in a lower frequency of potential adverse side effects associated with long-term administration of such therapeutic agents or methods. Thus, another indication of the compositions in this invention is to reduce adverse side effects of prior-art drugs or methods used to control optic nerve degeneration, such as the development of cataracts with long-acting anticholinesterase agents including demecarium, echothiophate, and isofluorophate.

In still another aspect, the present invention provides a method for preparing a composition for the treatment or control of an ocular neurodegenerative condition in a subject, which has an etiology in inflammation. The method comprises combining at least a PKC-θ inhibitor with a pharmactically acceptable carrier. The method may further include adding one or more pharmaceutically acceptable additives for providing certain desirable properties to the composition.

In one embodiment, a composition of the present invention is prepared to have a form of an emulsion, suspension, or dispersion. In another embodiment, the suspension or dispersion is based on an aqueous solution. For example, a composition of the present invention can comprise sterile saline solution.

A composition of the present invention can avoid one or more of the side effects of glucocorticoid therapy.

Glucocorticoids (“GCs”) are among the most potent drugs used for the treatment of allergic and chronic inflammatory diseases. However, as mentioned above, long-term treatment with GCs is often associated with numerous adverse side effects, such as diabetes, osteoporosis, hypertension, glaucoma, or cataract. These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β), IL-2, IL-3, IL-6, IL-11, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol., Vol. 94, 557-572 (1998). On the other hand, there is persuasive evidence that the synthesis of IκB kinases, which are proteins having inhibitory effects on the NF-κB pro-inflammatory transcription factors, is increased by GCs. These pro-inflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schäicke et al., Pharmacol. Ther., Vol. 96, 23-43 (2002). Thus, while the transactivation of certain genes by GCs produces beneficial effects, the transactivation of other genes by the same GCs can produce undesired side effects. Therefore, in another aspect, the present invention provides pharmaceutical compositions for the treatment, reduction, alleviation, or amelioration of a pathological condition having an etiology in inflammation, which compositions avoid generation of one or more adverse side effects of GCs.

In one aspect, an adverse side effect of GCs is selected from the group consisting of glaucoma, cataract, hypertension, hyperglycemia, hyperlipidemia (increased levels of triglycerides), and hypercholesterolemia (increased levels of cholesterol). In one embodiment, a level of said at least an adverse side effect is determined at about one day after said compounds or compositions are first administered to, and are present in, said subject. In another embodiment, a level of said at least an adverse side effect is determined about 30 days after said compounds or compositions are first administered to, and are present in, said subject. Alternatively, a level of said at least an adverse side effect is determined about 2, 3, 4, 5, or 6 months after said compounds or compositions are first administered to, and are present in, said subject.

In another aspect, said at least a prior-art glucocorticoid used to treat or reduce the same condition or disorder is administered to said subject at a dose and a frequency sufficient to produce the same beneficial effect on said condition or disorder as a compound or composition of the present invention after about the same elapsed time.

In still another aspect, said at least a prior-art glucocorticoid is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof. In one embodiment, said at least a prior-art glucocorticoid is selected from the group consisting of dexamethasone, prednisone, prednisolone, methylprednisolone, medrysone, triamcinolone, loteprednol etabonate, physiologically acceptable salts thereof, combinations thereof, and mixtures thereof. In another embodiment, said at least a prior-art glucocorticoid is acceptable for ophthalmic uses.

### Testing for Potential Side Effects

PKC-θ inhibitors are not expected to generate side effects that have been seen with glucocorticoid therapy. However, such effects may still be assessed by a test disclosed below. One of the most frequent undesirable actions of a glucocorticoid therapy is steroid diabetes. The reason for this is the stimulation of gluconeogenesis in the liver by the induction of the transcription of hepatic enzymes involved in gluconeogenesis and metabolism of free amino acids that are produced from the degradation of proteins (catabolic action of glucocorticoids). A key enzyme of the catabolic metabolism in the liver is the tyrosine aminotransferase (“TAT”). The activity of this enzyme can be determined photometrically from cell cultures of treated rat hepatoma cells. Thus, the gluconeogenesis by a glucocorticoid can be compared to that of a PKC-θ inhibitor by measuring the activity of this enzyme. For example, in one procedure, the cells are treated for 24 hours with the test substance (a PKC-θ inhibitor or a glucocorticoid), and then the TAT activity is measured. The TAT activities for the selected PKC-θ inhibitor and glucocorticoid are then compared. Other hepatic enzymes can be used in place of TAT, such as phosphoenolpyruvate carboxykinase, glucose-6-phosphatase, or fructose-2,6-biphosphatase. Alternatively, the levels of blood glucose in an animal model may be measured directly and compared for individual subjects that are treated with a glucocorticoid for a selected condition and those that are treated with a PKC-θ inhibitor for the same condition.

Another undesirable result of glucocorticoid therapy is increased IOP in the subject. IOP of subjects treated with a glucocorticoid or a PKC-θ inhibitor for a condition may be measured directly and compared.

Benefits of a composition of the present invention for neuroprotection can be determined, judged, estimated, or inferred by conducting assays and measurements, for example, to determine: (1) the protection of nerve cells from glutamate induced toxicity; and/or (2) the neural protection in a nerve crush model of mechanical injury. Non-limiting examples of such assays and measurements are disclosed in U.S. Pat. No. 6,194,415; which is incorporated herein by reference.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising at least a PKC-θ inhibitor; wherein said at least a PKC-θ inhibitor is present at a concentration such that the composition is capable of treating or controlling an eye condition, disorder, or disease in a subject, wherein said condition, disorder, or disease has an inflammatory component.

2. The composition of claim 1, further comprising an anti-inflammatory medicament.

3. The composition of claim 1, wherein said condition, disorder, or disease is selected from the group consisting of diabetic retinopathy (“DR”), wet age-related macular degeneration (“AMD”), dry AMD, diabetic macular edema (“DME”), posterior uveitis, anterior uveitis, intermediate uveitis, panuveitis, corneal inflammatory diseases, non-allergic conjunctivitis, keratoconjunctivitis, endophthalmitis, ocular neurodegeneration, glaucoma, optic neuritis, retinitis pigmentosa, and combinations thereof.

4. The composition of claim 1, wherein said at least a PKC-θ inhibitor comprises rottlerin.

5. The composition of claim 3, wherein said at least a PKC-θ inhibitor is selected from the group consisting of antibodies to PKC-θ.

6. The composition of claim 5, wherein said PKC-θ is human PKC-θ.

7. The composition of claim 3, wherein said at least a PKC-θ inhibitor comprises a PKC-θ antisense nucleic acid sequence.

8. The composition of claim 3, wherein said at least a PKC-θ inhibitor comprises a PKC-θ siNA, siRNA, dsRNA, miRNA, shRNA, or combination thereof.

9. The composition of claim 3, wherein said at least a PKC-θ inhibitor is present in an amount in a range from about 0.0001 to about 10 percent by weight of said composition.

10. The composition of claim 2, wherein said anti-inflammatory medicament comprises a material selected from the group consisting of non-steroidal anti-inflammatory drugs, DIGRAs, peroxisome proliferator-activated receptor (“PPAR”) ligands, and combinations thereof.

11. The composition of claim 1, further comprising a medicament selected from the group consisting of immunosuppressants, cyclooxygenase-2 inhibitors, DMARDS (disease-modifying anti-rheumatic drugs), anti-cell adhesion molecules, and combinations thereof.

12. The composition of claim 11, wherein said immunosuppressants are selected from the group consisting of cyclosporine, tacrolimus, rapamycinazathioprine, 6-mercaptopurine, and combinations thereof.

13. The composition of claim 1, wherein the composition has a pH in a range from about 5 to about 8.

14. A composition comprising: (a) at least a PKC-θ inhibitor; and (b) an additional medicament selected from the group consisting of NSAIDs, DIGRAs, VEGF inhibitors, PPAR ligands, immunosuppressants, cyclooxygenase-2 inhibitors, DMARDS, anti-cell adhesion molecules, and combinations thereof; wherein the composition is capable of treating or controlling: (i) an eye condition, disorder, or disease, or (ii) a degeneration of a component of an optic nerve system; wherein each of said at least a PKC-θ inhibitor and said additional medicament, when present, is present in an amount from about 0.0001 to about 5 percent by weight of said composition; and said composition has a pH of about 5-8.

15. A composition comprising: (a) at least a PKC-θ inhibitor; and (b) an IOP-lowering; wherein the composition is capable of treating or controlling a degeneration of a component of an optic nerve system; wherein each of said at least a PKC-θ inhibitor and said additional medicament, when present, is present in an amount from about 0.0001 to about 5 percent by weight of said composition; and said composition has a pH of about 5-8.

16. A method for treating or controlling: (i) an eye condition, disorder, or disease, which has an inflammatory component, or (ii) a degeneration of a component of an optic nerve system in a subject, the method comprising administering to an environment of an affected eye a pharmaceutically effective amount of a composition that comprises at least a PKC-θ inhibitor, wherein said composition is administered at a frequency effective to provide said treating or controlling.

17. The method of claim 16, wherein said condition, disorder, or disease is selected from the group consisting of DR, wet AMD, dry AMD, DME, posterior uveitis, anterior uveitis, intermediate uveitis, panuveitis, corneal inflammatory diseases, non-allergic conjunctivitis, keratoconjunctivitis, endophthalmitis, ocular neurodegeneration, glaucoma, optic neuritis, retinitis pigmentosa, and combinations thereof.

18. The method of claim 17, wherein said at least a PKC-θ inhibitor comprises an antibody to PKC-θ.

19. The method of claim 18, wherein said PKC-θ is human PKC-θ.

20. The method of claim 17, wherein said at least a PKC-θ inhibitor comprises a PKC-θ antisense nucleic acid molecule.

21. The method of claim 17, wherein said at least a PKC-θ inhibitor comprises a PKC-θ siNA, siRNA, dsRNA, miRNA, shRNA, or combination thereof.

22. The method of claim 17, wherein said at least a PKC-θ inhibitor is present in an amount in a range from about 0.0001 to about 10 percent by weight of said composition.

23. The method of claim 17, wherein the composition further comprises an additional medicament selected from the group consisting of NSAIDs, DIGRAs, VEGF inhibitors, PPAR ligands, immunosuppressants, cyclooxygenase-2 inhibitors, DMARDS, anti-cell adhesion molecules, and combinations thereof; wherein said additional medicament is present in an amount from about 0.0001 to about 5 weight percent.

24. A method for treating or controlling a degeneration of a component of an optic nerve system in a subject, the method comprising administering to an environment of an affected eye a pharmaceutically effective amount of a composition that comprises at least a PKC-θ inhibitor and an IOP-lowering medicament, wherein said composition is administered at a frequency effective to provide said treating or controlling.

25. A method for preparing a composition for treating or controlling: (i) an eye condition, disorder, or disease, or (ii) a degeneration of a component of an optic nerve system in a subject, the method comprising combining at least a PKC-θ inhibitor with a pharmaceutically acceptable carrier; wherein said at least a PKC-θ inhibitor is present at a concentration such that the composition is capable of treating or controlling said condition, disorder, disease, or degeneration in a subject.

26. The method of claim 25, further comprising adding a medicament selected from the group consisting of NSAIDs, DIGRAs, VEGF inhibitors, PPAR ligands, immunosuppressants, cyclooxygenase-2 inhibitors, DMARDS, anti-cell adhesion molecules, and combinations thereof to the composition.
